SEQUENCE LISTING (24 sequences)

SEQ ID NO: 1 - DNA, 23 bp, Artificial Sequence
  HBV-DNA Sense
atcctgctgctatgcctcatctt

SEQ ID NO: 2 - DNA, 23 bp, Artificial Sequence
  HBV-DNA AntiSense
acagtgggggaaagccctacgaa

SEQ ID NO: 3 - DNA, 31 bp, Artificial Sequence
  HBsAg Sense
gcacacggaattccgaggactggggaccctg

SEQ ID NO: 4 - DNA, 34 bp, Artificial Sequence
  HBsAg AntiSense
gacaccaagcttggttagggtttaaatgtatacc

SEQ ID NO: 5 - DNA, 22 bp, Artificial Sequence
  HBeAg Sense
acctcaccatactgcactcagg

SEQ ID NO: 6 - DNA, 21 bp, Artificial Sequence
  HBeAg AntiSense
ggctggaggagtgcgaatcca

SEQ ID NO: 7 - DNA, 25 bp, Artificial Sequence
  HBcAg Sense
accatggacattgacccttataaag

SEQ ID NO: 8 - DNA, 25 bp, Artificial Sequence
  HBcAg AntiSense
aggatccaacaacagtagtttccgg

SEQ ID NO: 9 - DNA, 22 bp, Artificial Sequence
  IFNAR Sense
gcggctcccagatgatggtcgt

SEQ ID NO: 10 - DNA, 22 bp, Artificial Sequence
  IFNAR AntiSense
tccatgacgtaagtagtgctgc

SEQ ID NO: 11 - DNA, 25 bp, Artificial Sequence
  STAT1 Sense
ccatggaaatcagacagtacctggc

SEQ ID NO: 12 - DNA, 25 bp, Artificial Sequence
  STAT1 AntiSense
ccttcacatttctgactttactgtc

SEQ ID NO: 13 - DNA, 27 bp, Artificial Sequence
  6-16 Sense
caagcttaaccgtttactcgctgctgt

SEQ ID NO: 14 - DNA, 28 bp, Artificial Sequence
  6-16 AntiSense
tgcggccgctgctggctactcctcacct

SEQ ID NO: 15 - DNA, 22 bp, Artificial Sequence
  MxA Sense
accagctgagcctgtccgaagc

SEQ ID NO: 16 - DNA, 22 bp, Artificial Sequence
  MxA AntiSense
ccggaccatatccgtcacggtg

SEQ ID NO: 17 - DNA, 22 bp, Artificial Sequence
  OAS Sense
tgatgggtccaccatccaggtg

SEQ ID NO: 18 - DNA, 22 bp, Artificial Sequence
  OAS AntiSense
cagcaggatgttcctgatggtc

SEQ ID NO: 19 - DNA, 22 bp, Artificial Sequence
  PKR Sense
tggctggtgatctttcagcagg

SEQ ID NO: 20 - DNA, 22 bp, Artificial Sequence
  PKR AntiSense
agagttgctttgggactcacac

SEQ ID NO: 21 - DNA, 25 bp, Artificial Sequence
  GAPDH AntiSense
tggaatcctgtggcatccatgaaac

SEQ ID NO: 22 - DNA, 25 bp, Artificial Sequence
  GAPDH AntiSense
taaaacgcagctcagtaacagtccg

SEQ ID NO: 23 - DNA, 20 bp, Artificial Sequence
  GAPDH Sense
ccatcaccatcttccaggag

SEQ ID NO: 24 - DNA, 20 bp, Artificial Sequence
  GAPDH AntiSense
cctgcttcaccaccttcttg

(19) United States
(12) Patent Application Publication
(10) Pub. No.: US 2013/0259835 A1
(43) Pub. Date: Oct. 3, 2013

(54) BIFIDOBACTERIUM ADOLESCENTIS SPM0212 HAVING ANTIVIRAL ACTIVITY FOR HEPATITIS B VIRUS, AND PHARMACEUTICAL COMPOSITION FOR PREVENTING AND TREATING HEPATITIS B COMPRISING THEREOF

(71) Applicants: Nam Joo HA, Seoul (KR); Kang Oh LEE, Seoul (KR); Do Kyung LEE, Seoul (KR)

(72) Inventors: Nam Joo HA, Seoul (KR); Kang Oh LEE, Seoul (KR); Do Kyung LEE, Seoul (KR)

(73) Assignee: SAHMYOOK UNIVERSITY INDUSTRY-ACADEMIC COOPERATION, Seoul (KR)

(21) Appl. No.: 13/910,575

(22) Filed: Jun. 5, 2013

Related U.S. Application Data
(62) Division of application No. 13/336,159, filed on Dec. 23, 2011.

(30) Foreign Application Priority Data
Dec. 16, 2011 (KR) 10-2011-0136760

(51) Int. Cl. A61K 35/74

(52) U.S. Cl. CPC A61K 35/745
USPC 424/93.4

(57) ABSTRACT

The present invention relates to a bifidobacterium adolescentis SPM0212 having antiviral activity for hepatitis B virus, and a pharmaceutical composition for preventing and treating hepatitis B comprising thereof. The bifidobacterium adolescentis SPM0212 or its fraction of the present invention shows antiviral activity for hepatitis B virus by blocking the production of antigen s of hepatitis B virus, and by inhibiting the expression of DNA, RNA and HBsAg of the HBV.

### CROSS-REFERENCE TO RELATED APPLICATION

This application is a Divisional of a U.S. patent application Ser. No. 13/336,159 filed on Dec. 23, 2011, which claims the benefit of priority from Korean Patent Application No. 10-2011-0136760, filed on Dec. 16, 2011, the contents of each of which are incorporated herein by reference.

### FIELD OF THE INVENTION

The present invention relates to a Bifidobacterium adolescentis SPM0212 having antiviral activity for hepatitis B virus, and a pharmaceutical composition for preventing and treating hepatitis B comprising thereof.

### BACKGROUND OF THE INVENTION

Hepatitis B virus (HBV) is a virus in the Hepadnaviridae family being specifically infected to a human body. Since the HBV was identified by Dr. Blumberg, it has been one of viruses damaging to humankind worldwide with the highest infection rate. Worldwide about 2,000 million people were infected, and about 350 million people among them are suffering from progressive liver diseases such as chronic hepatitis B, cirrhosis and liver cancer (Kim, 2010; Merican et al., 2000; Kao and Chen, 2002; Kim et al., 2010).

Because the HBV has broad clinical spectrum from healthy carrier state to intractable hepatitis and chronic hepatitis, it is being classified to an important disease around the world, and is widely spreading particularly to orient and Africa region. Korea is also one of the countries having high hepatitis B prevalence rate and it is becoming a global public health problem (Woo et al., 1999; Joo et al., 2005; Lavanchy, 2004, Kang et al., 1999).

The chronic active hepatitis develops and causes cirrhosis, liver cancer and the like, and can become worse to severe acute hepatitis accompanying liver failure (Brechot., J. Hepatol., 4, 269-279, 1987). Most of the chronic hepatitis patients show continuous or active hepatitis symptoms while part of them does not show the symptoms.

The viral hepatitis attack mechanism or inducing factor has not yet been found clearly. Since some evidences that the liver damage caused by the hepatitis B virus is mediated by host immune cells has been suggested (Milich, D. R. et al., J. Immunol., 143, 3141-3147, 1989), studies for a virus antigen causing liver cell necrosis , immune reaction of certain B cell an T cell, and direct cellular attack of the disease of the virus protein or cancer attack mechanism were intensively achieved (R. Zschke, O. et al., Nature, 348, 252-254, 1990). However, still the cause of the clinical pathways, which are variously expressed per patient, are not clear.

The purpose of chronic hepatitis B treatment is to prevent the progress to cirrhosis or liver cancer by inhibiting or removing HBV proliferation in the long term or permanently, and thus to reduce the death rate (Liaw et al., 2008; Lok and McMahon, 2007). Studies for treating the chronic hepatitis B has been continuously achieved, but in general, it is still difficult to completely treat the virus until now (Kim et al, 2010).

Up to now, as a result of treating the hepatitis B virus by many methods using interferon, nucleic acid derivative or immune regulation substance, interferon-α is accepted as a hepatitis therapeutic agent in the states because it has almost only the said effects. The nucleic acid derivative and the like, which were developed for the first time and has been used, inhibited the virus at first but many problems, particularly of deterioration of the patient condition, caused by mutation creation and tolerance as time goes on has been reported. The interferon-α was tested by Sculled et al., as the chronic hepatitis B therapeutic agent in the middle of 1970s for the first time, and it was reported that it has an effect of inhibiting the HBV replication. Then, as the interferon-α has been produced by gene recombination method, its use significantly increased (J. Infect. Dis., 143: 772-783, 1981). However, when the interferon-α had been administered 3 times a week for at least 3 months, it only showed treatment effect of inhibiting the virus replication with average 20% patients, but did not show the continuous inhibitory effect. Therefore, its therapeutic effect was low because patients by vertical transmission between mother and child, which is particularly abundant to oriental people, showed resistance to the interferon-α. Further, recently, it was reported that only 50% or less of the acute or chronic patients infected by the hepatitis B virus were suitable for treating the interferon-α, and the effect of the treatment method of the interferon can be expected only when the amount of various enzymes produced in the liver (AST, SGOT, ALT. SGPT and the like) by the affect to the virus increases.

Lamivudine (LMV), which has been used as an oral antiviral medicine for hepatitis B long since, is an oral nucleoside derivative preventing viral DNA replication by competitively inhibiting a reverse transcriptase of HBV, and broadly has used as a primary therapeutic agent by effectively inhibiting proliferation of HBV DNA and by preventing progress of liver diseases (Song, 2010; Kim et al., 2009; Jang et al., 2001). However, because it only inhibits a viral reverse transcription process, it can't destroy the HBV DNA of covalently closed circular (ccc) form existing in a liver cell, and because it also can't inhibit a transcription process of a viral mRNA, which is transcribed from the cccDNA, it can't completely kill the HBV (Doong et al., 1991; Chang et al., 1992; Jang et al., 2001). Therefore, it should be ultimately used for a long time or for a lifetime because the disease can come back and the liver function can take a turn for the worse when the treatment is stopped (Dienstag et al., 1999; Lai et al., 1998). However, it has been reported that the long-term use of this oral antiviral medicine may increase the incidence of tolerance, and have effect on the actions of antiviral medicines having other tolerance (Locamini et al., 2004; Lai et al., 2003; Dienstag et al., 2003a; Lok et al., 2003; Andreone et al., 2004; Liaw et al., 2004; Yuen et al., 2007).

Thus, a study for substitute medicines showing antiviral effect without any side effect is an important subject.

### SUMMARY OF THE INVENTION

Accordingly, it is an object of the present invention to provide a bifidobacterium adolescentis SPM0212 showing anti-hepatitis B viral activity as a novel therapeutic agent for hepatitis B.

It is another object of the present invention to provide a pharmaceutical composition for preventing and treating hepatitis B comprising the bifidobacterium adolescentis SPM0212 or culture thereof.

It is further another object of the present invention to provide a health functional food for preventing and improving hepatitis B comprising the bifidobacterium adolescentis SPM0212 of or culture thereof.

In accordance with one aspect of the present invention, there is provided a bifidobacterium adolescentis SPM0212 (Accession No.: KCTC 18120P) showing antiviral activity for hepatitis B virus.

In the present invention, bacteria were separated from 20-30 years old healthy adults having normal eating habit, and bacteria showing antiviral activity for hepatitis B virus were selected by enzyme-linked immune sorbent assay (ELISA). The selected bacteria were identified by nucleotide sequence analysis of 16S rRNA, PCR-RAPD analysis, and analysis of morphological, cultural and biochemical characteristics. As a result, it was confirmed that the lactobacillus separated in the present invention is a “bifidobacterium adolescentis”.

The present inventors named the lactobacillus ‘bifidobacterium adolescentis SPM0212’, and submitted to biological resource center of Korea research institute of bioscience and biotechnology on Aug. 18, 2006 (Accession No.: KCTC 18120P).

In accordance with another aspect of the present invention, there is provided a pharmaceutical composition for preventing and treating hepatitis B comprising the bifidobacterium adolescentis SPM0212 (Accession No.: KCTC 18120P) or culture thereof.

In accordance with further another aspect of the present invention, there is provided a health functional food for preventing and improving hepatitis B comprising the bifidobacterium adolescentis SPM0212 of or culture thereof.

In the present invention, the bifidobacterium adolescentis SPM0212 is characterized by inhibiting the production of s antigen of hepatitis B virus, and the expression of DNA, RNA and HBsAg of the HBV.

### Advantageous Effects of the Invention

The bifidobacterium adolescentis SPM0212 of the present invention or culture thereof inhibits the production of s antigen of hepatitis B virus, and the expression of DNA, RNA and HBsAg of the HBV so as to show the antiviral activity for the HBV, and also can be usefully used as a pharmaceutical composition for preventing and treating hepatitis or a health functional food for preventing and improving hepatitis B because it is a non-toxic lactobacillus.

### BRIEF DESCRIPTION OF DRAWINGS

The above and other objects and features of the present invention will become apparent from the following description of the invention taken in conjunction with the following accompanying drawings, which respectively show:

FIG. 1 presents a diagram of Bifidobacteriaceae family.

FIG. 2 presents results of ELISA of HBsAg using a reagent for diagnosing hepatitis A and B (Enzygnost HBsAg monoclonal, Behring, Germany).

FIG. 3 presents results of ELISA of HBsAg for the separated 6 kinds of lactobacillus using a reagent for diagnosing hepatitis A and B (Enzygnost HBsAg monoclonal, Behring, Germany).

FIG. 4 presents a result of ELISA of HBsAg using a reagent for diagnosing hepatitis A and B (Enzygnost HBsAg monoclonal, Behring, Germany) according to a combined treatment of lamivudine and the bifidobacterium adolescentis SPM0212.

FIG. 5 presents results of measuring the HBV-DNA expression according to the treatment of the bifidobacterium adolescentis SPM0212 of the present invention.

FIGS. 6 and 7 present results of measuring the HBV RNA level of an intracellular genome and its effect on the HBsAg gene expression according to the treatment of the bifidobacterium adolescentis SPM0212 of the present invention, respectively.

FIGS. 8 to 13 present results of measuring the effect of the HBV on the expression of IFNAR, STAT1, 6-16, M×A, PKR and OAS according to the treatment of lactobacillus, respectively.

FIG. 14 presents results of measuring the cellular toxicity of the separated lactobacillus.

FIG. 15 presents a result of analyzing an antivirus active material of the bifidobacterium adolescentis SPM0212 of the present invention.

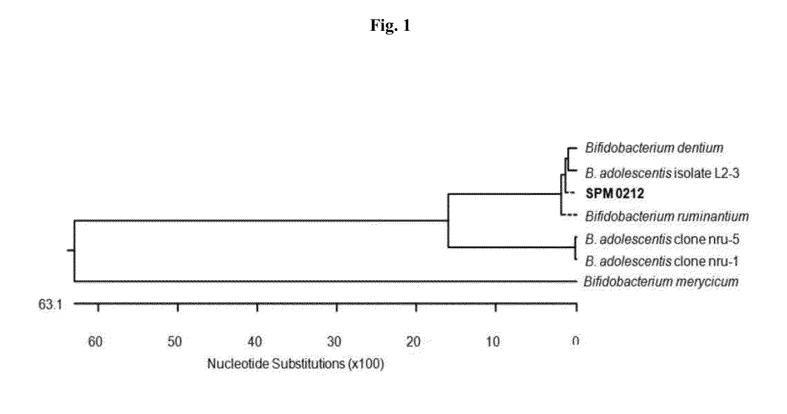

### DETAILED DESCRIPTION OF THE INVENTION

Hereinafter, the following Examples are intended to illustrate the present invention without limiting its scope.

### Example 1

### Selection of Lactobacillus Strain having Anti-HBV Activity

Fifteen kinds bacteria (ten kinds of bifidobacterium adolescentis (B. adolescentis), three kinds of bifidobacterium longum (B. longum) and two kinds of bifidobacterium B. psedcatenulatum) as listed in Table 1 from 20-25 years old healthy Koreans having eating habit mainly of vegetarian diet were identified.

TABLE 1

Origin

| | | | |
|---|---|---|---|
| Strains | Kind | Sex | Age |

Bifidobacterium adolescentis

| | | | |
|---|---|---|---|
| SPM0212 | Human | Female | 21 |
| SPM0214 | Human | Female | 21 |
| SPM0308 | Human | Female | 22 |
| SPM1005 | Human | Male | 25 |
| SPM1307-A | Human | Male | 24 |
| SPM1601 | Human | Male | 20 |
| SPM1604 | Human | Male | 20 |
| SPM1605 | Human | Male | 20 |
| SPM1606 | Human | Male | 20 |
| SPM1608 | Human | Male | 20 |

Bifidobacterium pseudocatenulatum

| | | | |
|---|---|---|---|
| SPM1204 | Human | Female | 22 |
| SPM1309 | Human | Male | 24 |

Bifidobacterium longum

| | | | |
|---|---|---|---|
| SPM1205 | Human | Female | 22 |
| SPM1206 | Human | Female | 22 |
| SPM1207 | Human | Female | 22 |

The separated Bifidobacterium spp. SPM strains were cultured using a General anaerobic Medium in a Bactron Anaerobic Chamber of 37° C. and, anaerobic condition (90% N₂, 5% H₂, 5% CO₂) for 48 hours. Then, the culture solution was centrifuged at 4,000 rpm for 10 min to separate supernatant and cells and they were collected, respectively. The cells were once washed with phosphate-buffered saline (PBS; pH 6.8) followed by resuspending in PBS, and fragmented using a sonicator under a condition of amplitude 100%, pulse on-60s, pulse off-60s and 6 cycle. The cell fragments and the culture supernatant were filtered through a 0.22 μm-sized syringe filter, and the filtrated solution was used in the following experiments.

### Test Example 1

### HepG 2, HepG 2.2.15 Cell Culture

In order to examine the antiviral activity for hepatitis virus, a stable cell line HepG2.2.15 prepared by transfecting HBV DNA to a liver cancer cell HepG2 (Sells et al., 1987) was distributed from Professor Park, Yun Gyu of Korea University College of Medicine and used as a HepG 2, HepG 2.2.15. cell line charing an s antigen of hepatitis B virus (HBsAg) and e antigen (HBeAg) to a medium because hepatitis B virus gene was inserted into the HepG 2.2.15 cell (human hepatoblastoma cell) derived from human liver tissue.

The HepG 2, HepG 2.2.15 cell was seeded to MEM medium (Gibco, USA) supplemented with 4 μg/ml gentamicin (Sigma, USA) and 10% heated fetal bovine serum (Gibco, USA) on a petridish having a diameter of 10 cm, and cultured in an incubator of 37° C. and 5% CO₂. When a mono cell layer was formed, trypsin was added thereto every 3 to 4 days for succeeding.

### Test Example 2

### Measuring Anti-Hepatitis B Virus Activity Using ELISA

In order to examine the anti-hepatitis virus activity for hepatitis B virus of the separated lactobacillus cell line, the lactobacillus was treated to the cultured HepG2, HepG2.2.15, and then the inhibitory effect on the s antigen production was watched for. When the hepatitis B virus is replicated, three major antigens (c, s, e antigens) are produced, wherein the c (core) antigen is a structural antigen crystal and the s (surface) antigen is an antigen crystal represented by a surface protein of the virus.

### Test Example 2-1

### ELISA of Hepatitis B virus s Antigen (HBsAg ELISA)

Enzyme-linked immune sorbent assay (ELISA) of the hepatitis B virus s antigen was performed using a reagent for diagnosing the hepatitis B antigen (Enzygnost HBsAg monoclonal, Behring, Germany).

After culturing the HepG 2.2.15 cells to the concentration of 2×10⁵ cells/well for 3 days using a 12-well plate, the culture supernatant was removed, and the cellular extract of the Bifidobacterium spp. SPM (1×10⁸ CFU) and various concentration of the culture supernatant was treated together with new medium. As a Comparative Example, a group treating the same amount of PBS to the HepG2.2.15 was used to the concentration of 125 μg/mL. The cells were culture for 24, 48 and 72 hours, respectively, each culture solution was obtained and quantified the HBsAg using GENEDIA HBsAg ELISA 3.0.

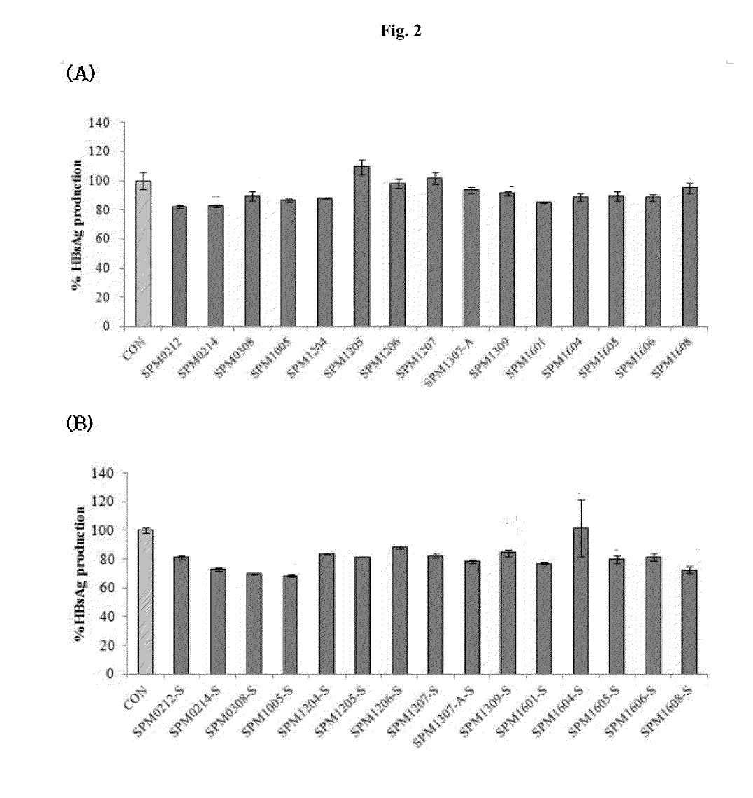

A sample 100 μl and a manufactured conjugate 25 μl were added to the provided 96-well plate followed by mixing well by lightly patting the frame, and reacted at 37° C. for 90 min. After the reaction was completed, the contents of each well were sucked out, and each well was five times washed with a washing solution. A manufactured substrate solution 100 id was added to each well and reacted at room temperature for 30 min. After the reaction was completed, a reaction stopper solution 100 μl was added to each well, and the absorbance was measured at 450 nm (reference wavelength: 620 nm) using an ELISA reader. The results were shown in FIG. 2.

As shown in FIG. 2, 5 kinds of bifidobacterium adolescentis (B. adolescentis) SPM0212, SPM0214, SPM0308, SPM1005 and SPM1601, and 1 kind of B. pseudocatenulatum SPM1204 which were expressed about 20% inhibitory effect at the cellular extract of 1×10⁶ CFU, were firstly selected.

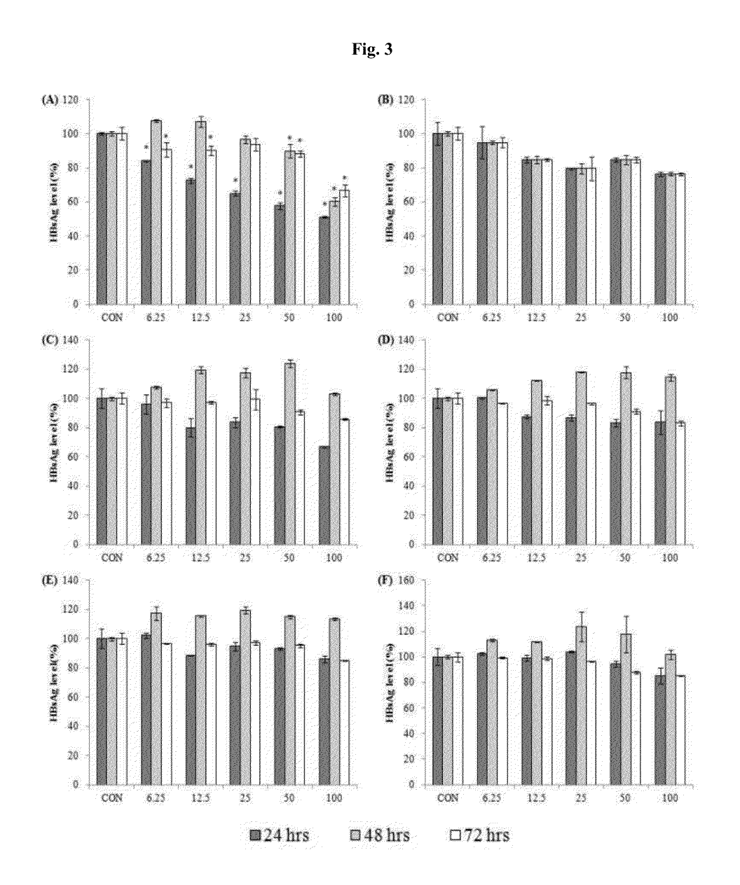

After increasing the concentrations of the selected 6 kinds of lactobacillus, cellular extract of 1×10⁸ CFU was sequentially diluted two times and treated to the HepG2.2.15 cell according to the concentration. The cells were culture for 24, 48 and 72 hours, respectively, and as described above, the HBsAg was quantified using GENEDIA HBsAg ELISA 3.0. The results were shown in FIG. 3.

In FIG. 3, (A) represents SPM0212, (B) represents B. adolescentis SPM0214, (C) represents B. adolescentis SPM0308, (D) represents B. adolescentis SPM1005, (E) represents B. pseudocatenulatum SPM1204 and (F) represents B. adolescentis SPM1601, and it was confirmed that the inhibitory effect on the HBV increases with the increased concentration when the B. adolescentis SPM0212 was treated.

### Test Example 2-2

### ELISA of Hepatitis B virus s Antigen (HBsAg ELISA) in Combination with Lamivudine

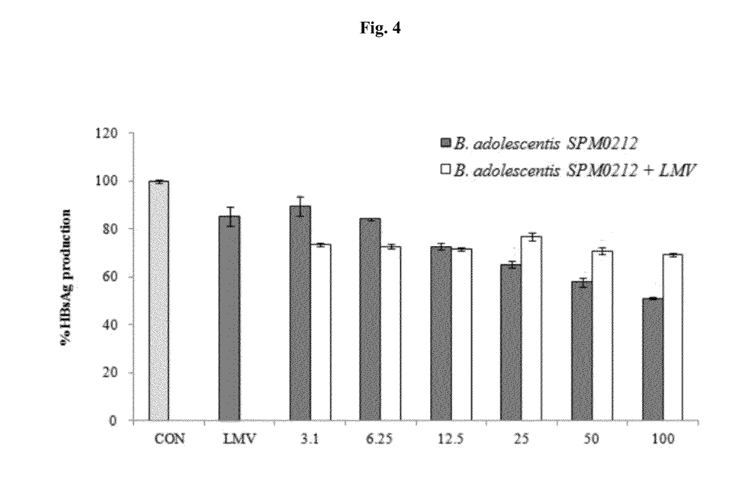

In order to found out the combination effect of the bifidobacterium adolescentis SPM0212, which showed the inhibitory effect on the hepatitis virus activity depending on the concentration in Test Example 2-1, and lamivudine, while changing the concontreation of the bifidobacterium adolescentis SPM0212 cell extract, lamivudine 125 μl/mL was added thereto. Then, as described in Test Example 2-1, the HBsAg was quantified using the GENEDIA HBsAg ELISA 3.0, and the results were shown in FIG. 4.

In FIG. 4, it was confirmed that when the bifidobacterium adolescentis SPM0212 was treated at low concentration, the HBsAg producing effect was better than that of treating the lamivudine only.

### Test Example 2-3

### Measuring DNA and Gene Expression Change of Hepatitis B Virus

The change in the HBV gene expression caused by the cellular extract of the bifidobacterium adolescentis SPM0212 selected in the above Test Example was analyzed.

After culturing the HepG2.2.15 cells to the concentration of 2×10⁵ cells/well (12-well plate) for 3 days, the culture supernatant was removed, and the cellular extract of the bifidobacterium adolescentis SPM0212 (1×10⁸ CFU) was treated to the various concentration (sequentially diluted two times) together with new medium.

In order to analyze DNA of the HBV, after culturing the cells for 24 hours, each cells and culture solutions were collected, and centrifugated at 1,200 rpm for 10 min to separate the culture solutions and the cells. Total DNA was extracted from the cells using QIAamp®DNA Mini and Blood Mini kit, and the extracted DNA was quantified to 25 ng/μl to be used as a template of real-time quantitative PCR(RT-qPCR) reaction. The used primers were listed in Table 2. Further, the DNA analysis of the HBV in the culture solution was performed using the culture solution 2 μl as a template (Lee at al., 2009b).

TABLE 2

| Target | | | Ann. Temp. | |
|---|---|---|---|---|
| Genes | Polarity | Nucleotide sequences | (° C.) | Cycles |
| HBV-DNA | Sense | ATC CTG CTG CTA TGC CTC | 60 | 34 |
| | | ATC TT | | |
| | Antisense | ACA GTG GGG GAA AGC CCT | | |
| | | ACG AA | | |
| HBsAg | Sense | GCA CAC GGA ATT CCG AGG | 65 | 45 |
| | | ACT GGG GAC CCT G | | |
| | Antisense | GAC ACC AAG CTT GGT TAG | | |
| | | GGT TTA AAT GTA TAC C | | |
| HBeAg | Sense | ACC TCA CCA TAC TGC ACT | 59 | 30 |
| | | CAG G | | |
| | Antisense | GGC TGG AGG AGT GCG AAT | | |
| | | CCA | | |
| HBcAg | Sense | ACC ATG GAC ATT GAC CCT | 58 | 45 |
| | | TAT AAA G | | |
| | Antisense | AGG ATC CAA CAA CAG TAG | | |
| | | TTT CCG G | | |
| IFNAR | Sense | GCG GCT CCC AGA TGA TGG | 57 | 27 |
| | | TCG T | | |
| | Antisense | TCC ATG ACG TAA GTA GTG | | |
| | | CTG C | | |
| STAT1 | Sense | CCA TGG AAA TCA GAC AGT | 58 | 45 |
| | | ACC TGG C | | |
| | Antisense | CCT TCA CAT TTC TGA CTT | | |
| | | TAC TGT C | | |
| 6-16 | Sense | CAA GCT TAA CCG TTT ACT | 56 | 30 |
| | | CGC TGC TGT | | |
| | Antisense | TGC GGC CGC TGC TGG CTA | | |
| | | CTC CTC ACC T | | |
| MxA | Sense | ACC AGC TGA GCC TGT CCG | 59 | 32 |
| | | AAG C | | |
| | Antisense | CCG GAC CAT ATC CGT CAC | | |
| | | GGT G | | |
| OAS | Sense | TGA TGG GTC CAC CAT CCA | 59 | 32 |
| | | GGT G | | |
| | Antisense | CAG CAG GAT GTT CCT GAT | | |
| | | GGT C | | |
| PKR | Sense | TGG CTG GTG ATC TTT CAG | 56 | 32 |
| | | CAG G | | |
| | Antisense | AGA GTT GCT TTG GGA CTC | | |
| | | ACA C | | |
| βActin | Sense | TGG AAT CCT GTG GCA TCC | 58 | 45 |
| | | ATG AAA C | | |
| | Antisense | TAA AAC GCA GCT CAG TAA | | |
| | | CAG TCC G | | |
| GAPDH | Sense | CCA TCA CCA TCT TCC AGG | 58 | 27 |
| | | AG | | |
| | Antisense | CCT GCT TCA CCA CCT TCT | | |
| | | TG | | |

The RT-qPCR reaction was performed using LightCycler® FastStart DNA Master SYBR Green I kit in LightCycler system. Relative gene expression data were calculated according to the method of Livak and Schmittgen, and housekeeping gene GAPDH was used as an internal control (Livak and Schmittgen, 2001).

Amount of target=2^{-ΔΔCT}

ΔΔC_{T}=(C_{T,Target}−C_{T,GAPDH})_{Sample}−(C_{T,Target}−C_{T,GAPDH})_{Control}

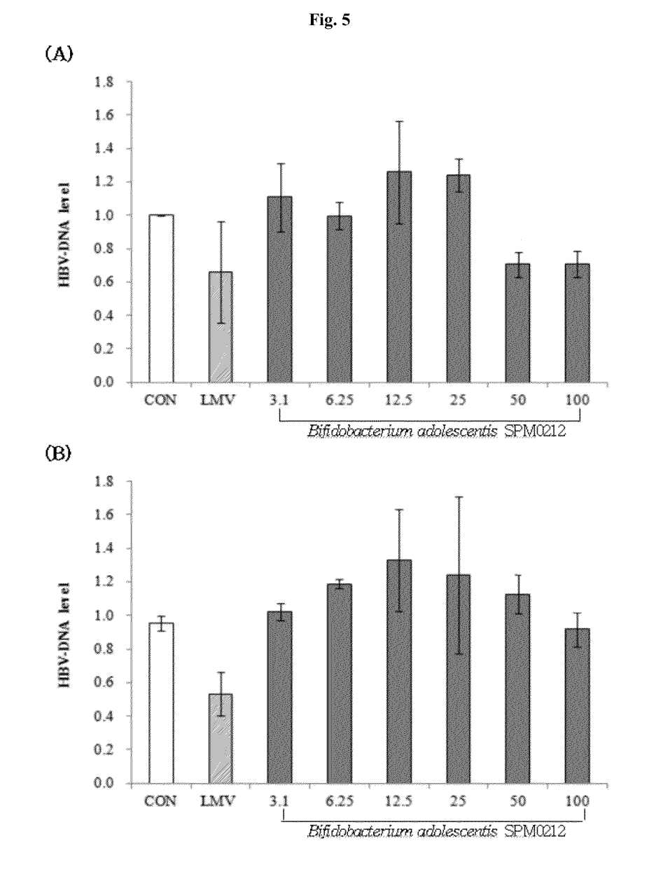

As shown in FIG. 5 B, in the HBV-DNA expression, DNA proliferation in a cell was not quite different from the control group, but as shown in FIG. 5 A, the level of HBV DNA secreted out of the cell was reduced up to 30%. It was confirmed that when the bifidobacterium adolescentis SPM0212 cell extract (1×10⁸ CFU) was treated, it was similar with the Comparative Example using the lamivudine of 125 μl/mL.

In order to find out the influence on the RNA level of an intracellular HBV genome of the HBV and the HBsAg gene expression, after sequentially diluting the bifidobacterium adolescentis SPM0212 (1×10⁸ CFU) cell extract and treating, RNA of the treated HepG2.2.15 cell was separated, and total RNA was extracted using RNeasy Mini kit. Then reverse transcription RT-qPCR analysis was performed.

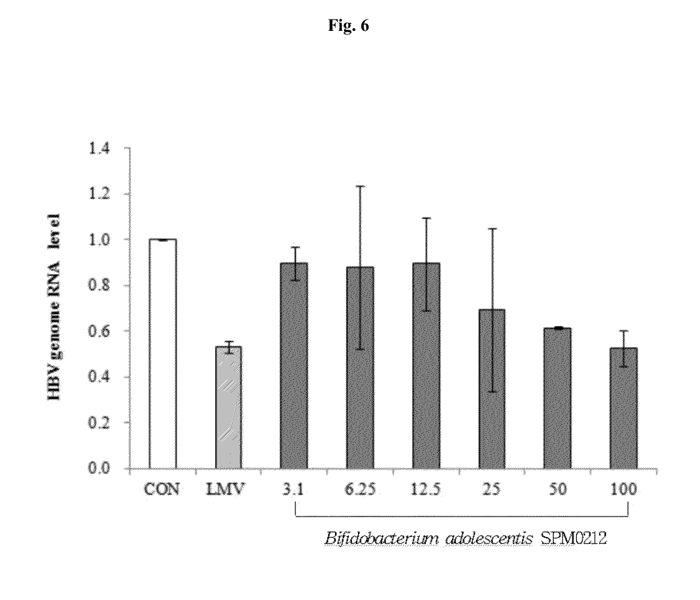

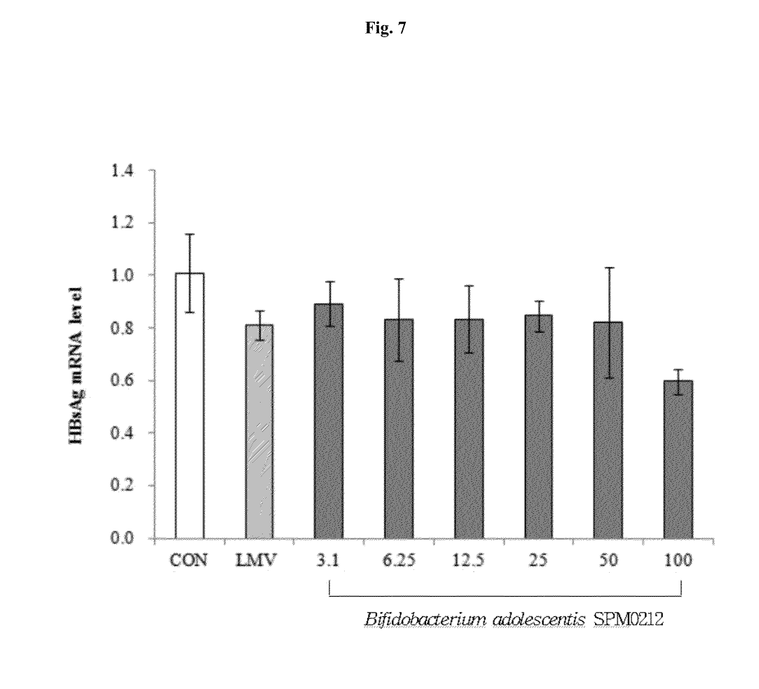

As shown in FIG. 6, the RNA level of the HBV genome was reduced up to about 50% as much as the Comparative Example using the lamivudine of 125 μl/mL, and as shown in FIG. 7, the HBsAg gene expression was inhibited up to 40%, and it was better than the inhibitory effect of the comparative Example using the lamivudine of 125 μl/mL.

### Test Example 3

### Analysis of Antiviral Pathway

In order to find out anti-viral pathway caused by the bifidobacterium adolescentis SPM0212 cell extract, Interferon (IFN)-mediated antiviral reaction pathway was observed.

Using the RNA extracted in Test Example 2-3, the gene expressions of factors related to the interferon (IFN)-mediated antiviral reaction pathway such as IFN-α receptor (IFNAR), IFN-α inducible genes (signal transducers and activators of transcription (STAT)1 and 6-16), and the expressions of genes coding antiviral effectors (myxovirus resistance A (M×A), 2′,5′-oligoadenylate synthetase (OAS) and protein kinase R(PKR)) were analyzed by reverse transcription RT-qPCR.

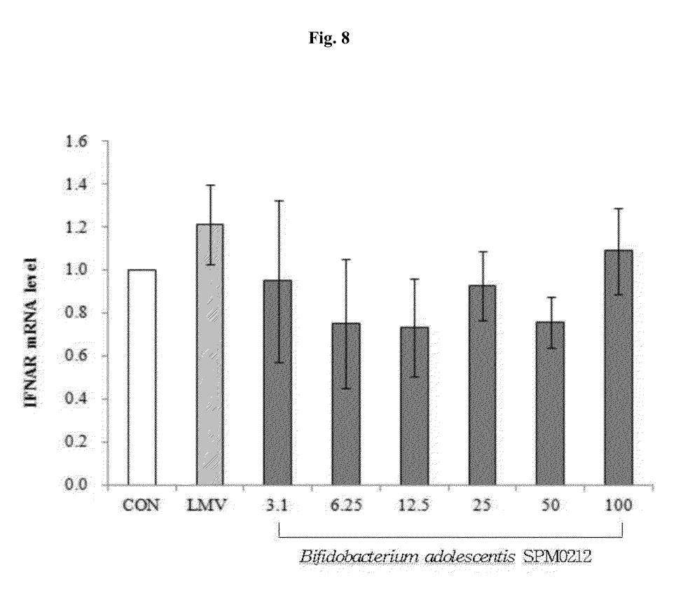

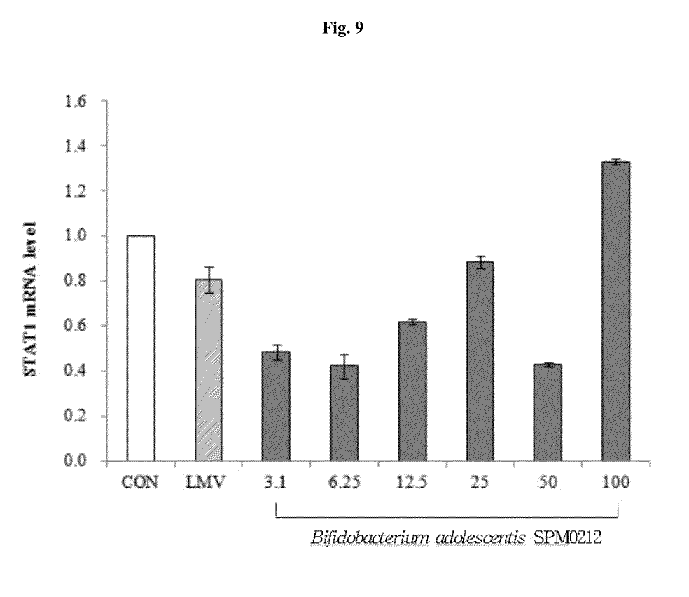

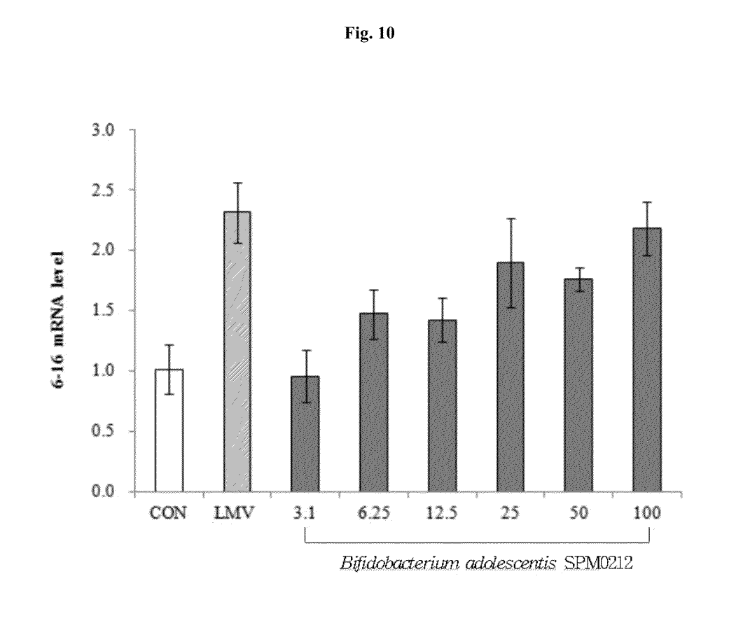

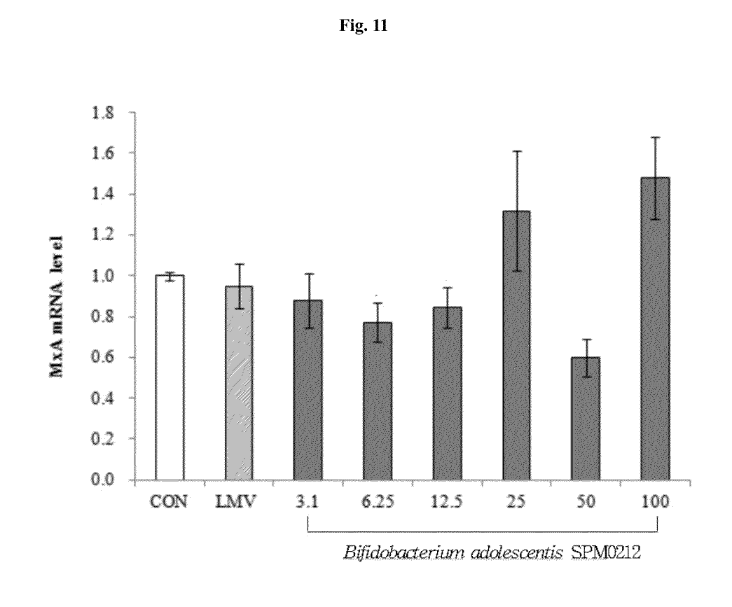

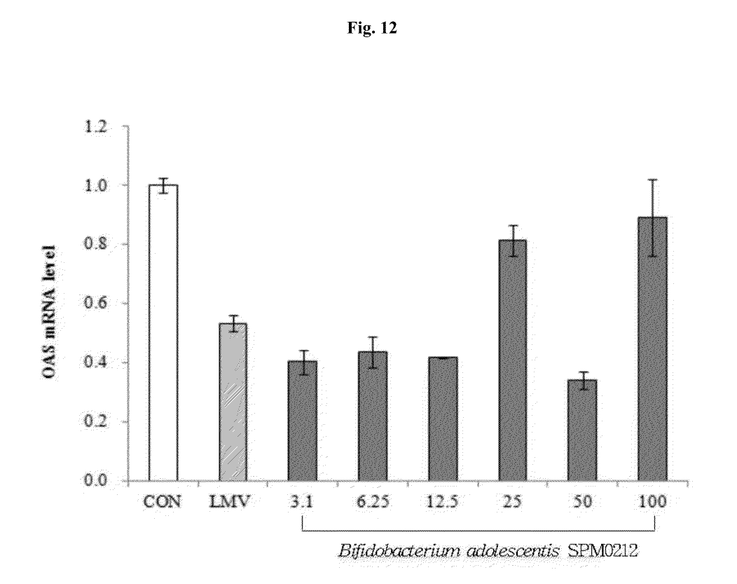

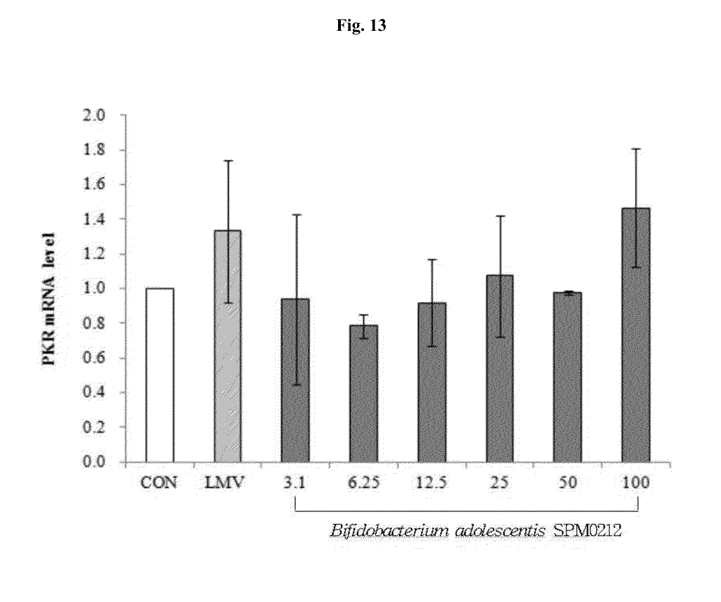

As a result, in FIGS. 8 to 13, B. adolescentis SPM0212 (1×10⁸ CFU) cell extract did not affect to the IFNAR expression, but increased 1.3 times and 2.1 times the expressions of IFN-a inducible genes, STAT1 and 6-16, respectively. Further, the expression of one of the antiviral effectors, which was produced from the STAT1 activation, M×A was 1.4 times increased. The expression of other antiviral effector, PKR was 1.5 times increased, but the significance was not observed, and the OAS expression was not significantly increased.

### Example 2

### Identification of Separated Lactobacillus Strain

In order to identify the Bifidobacterium spp. separated in Example 1, fructose-6-phosphate phosphoketolase (F6PPK) activity assay (Ahn, 2005a) and 16S rRNA sequencing were performed (requested to Bioleaders (Korea)), and the results were listed in Table 3.

TABLE 3

| | B. adolescentis SPM | | | | | B. longum SPM | | | |
|---|---|---|---|---|---|---|---|---|---|
| Sugar | 0212 | 0308 | 1005 | 1307-A | 1601 | 1205 | 1206 | 1207 | SPM1309 |
| L-Arabinose | − | − | + | − | − | − | + | + | − |
| D-Ribose | − | − | − | − | − | − | − | − | − |
| Xylose | + | − | + | − | + | − | − | + | − |
| Galactose | + | + | + | + | + | + | + | + | + |
| Fructose | + | + | + | − | + | − | + | − | − |
| Mannose | − | − | − | − | + | − | − | − | − |
| Mannitol | − | − | + | − | − | − | − | − | − |
| Sorbitol | − | − | − | − | − | − | − | − | − |
| Salicine | − | − | + | − | − | − | − | − | − |
| Cellobiose | − | − | − | − | + | − | − | − | − |
| Maltose | + | − | − | − | − | − | − | + | − |
| Lactose | + | − | + | + | − | − | + | + | + |
| Melibiose | + | + | + | + | + | + | + | + | + |
| Saccharose | + | − | + | + | + | − | − | + | + |
| Trehalose | + | − | + | − | − | − | − | − | − |
| Inuline | − | − | + | − | − | − | − | + | − |
| Melezitose | + | − | + | − | − | − | − | + | − |
| Raffinose | + | − | − | − | + | − | − | + | − |
| Starch | + | − | + | − | + | − | − | − | − |
| Gluconate | + | − | − | − | − | − | − | − | − |

The all separated Bifidobacterium Lactobacillus strains had Galactose and Melibiose fermenting abilities, and only B. adolescentis SPM0212 strain also had Gluconate fermenting ability.

### Example 3

### Antibiotics Sensitivity Analysis of Bifidobacterium spp. SPM

Antibiotics sensitivity test was performed for comparative analysis of phenotypes of the lactobacillus strain separated in Example 1 and standard strains purchased from KCTC. In order to find out the minimum inhibitory concentrations (MICs) of 18 kinds of antibiotics (ampicillin, mupirocin, amoxicillin/clavulanic acid, oxacillin, cefonicid, cefazolin, ceftazidime, cefotaxime, ceftriaxone, tigecyclin, streptomycin, vancomycin, teicoplanin, daptomycin, linezolid, clindamycin, quinupristin/dalfopristin and tobramycin), agar dilution method was performed according to the Clinical and Laboratory Standards Institute (CLSI, 2003), and the results were listed in Tables 4 and 5.

TABLE 4

| | Minimum inhibitory concentrations (MICs) (μl/mL) | | | | | | | | |
|---|---|---|---|---|---|---|---|---|---|
| Strains | AMP | MUP | AMC | OXA | CID | CFZ | CAZ | CTX | CRO |

Bifidobacterium adolescentis

| | | | | | | | | | |
|---|---|---|---|---|---|---|---|---|---|
| KCTC3352 | ≦0.06 | >128 | 0.5 | 2 | 16 | 4 | 16 | 2 | 4 |
| SPM0212 | ≦0.06 | >128 | 0.5 | 1 | 8 | 2 | 2 | 0.25 | 4 |
| SPM0214 | ≦0.06 | >128 | 0.5 | 1 | 4 | 1 | 1 | 0.12 | 0.12 |
| SPM0308 | ≦0.06 | >128 | 0.5 | 1 | 4 | 1 | 1 | 0.25 | 0.12 |
| SPM1005 | ≦0.06 | >128 | 0.5 | 1 | 8 | 2 | 2 | 0.25 | 0.25 |
| SPM1307-A | ≦0.06 | >128 | 0.25 | 0.5 | 2 | 0.5 | 0.5 | 0.12 | 0.12 |
| SPM1601 | ≦0.06 | >128 | 0.25 | 0.5 | 4 | 0.5 | 1 | 0.25 | 0.12 |
| SPM1604 | ≦0.06 | ≦0.06 | ≦0.06 | ≦0.06 | ≦0.06 | ≦0.06 | ≦0.06 | ≦0.06 | ≦0.06 |
| SPM1605 | ≦0.06 | >128 | 0.25 | 0.5 | 4 | 0.5 | 1 | 0.25 | 0.12 |
| SPM1606 | ≦0.06 | >128 | 0.25 | 0.5 | 2 | 0.5 | ≦0.06 | 0.12 | ≦0.06 |
| SPM1608 | ≦0.06 | >128 | 0.25 | 0.5 | 2 | 0.5 | 1 | 0.12 | 0.12 |

Bifidobacterium pseudocatenulatum

| | | | | | | | | | |
|---|---|---|---|---|---|---|---|---|---|
| KCTC3223 | ≦0.06 | >128 | 0.5 | 2 | 8 | 4 | 4 | 2 | 4 |
| SPM1204 | ≦0.06 | >128 | 2 | 4 | 8 | 8 | 4 | 2 | 2 |
| SPM1309 | ≦0.06 | >128 | 1 | 4 | 16 | 4 | 2 | 2 | 2 |

Bifidobacterium longum

| | | | | | | | | | |
|---|---|---|---|---|---|---|---|---|---|
| KCTC3128 | ≦0.06 | >128 | 1 | 2 | 8 | 4 | 4 | 2 | 2 |
| SPM1205 | 0.12 | >128 | 2 | 8 | 128 | 32 | 8 | 2 | 4 |
| SPM1206 | 0.25 | >128 | 1 | 8 | 128 | 32 | 16 | 4 | 4 |
| SPM1207 | ≦0.06 | >128 | 2 | 4 | 64 | 32 | 4 | 4 | 4 |

AMP: ampicillin,
MUP: mupirocin,
AMC: amoxicillin/clavulanic acid,
OXA: oxacillin,
CID: cefonicid,
CFZ: cefazolin,
CAZ: ceftazidime,
CTX: cefotaxime,
CRO: ceftriaxone

TABLE 5

| | Minimum inhibitory concentrations (MICs) (μl/Ml) | | | | | | | | |
|---|---|---|---|---|---|---|---|---|---|
| Strains | TIG | STR | VAN | TEC | DAP | LNZ | CLI | QDA | TOB |

Bifidobacterium adolescentis

| | | | | | | | | | |
|---|---|---|---|---|---|---|---|---|---|
| KCTC3352 | 0.5 | 128 | 2 | 2 | 16 | 8 | 4 | 1 | >128 |
| SPM0212 | ≦0.06 | 128 | 2 | 2 | 4 | 8 | 4 | 0.5 | >128 |
| SPM0214 | 0.25 | 32 | 2 | 2 | 2 | 0.25 | 32 | ≦0.06 | 32 |
| SPM0308 | ≦0.06 | 32 | 2 | 2 | 0.25 | 2 | 32 | ≦0.06 | 32 |
| SPM1005 | 0.5 | 32 | 2 | 1 | 1 | 4 | 32 | 0.25 | 32 |
| SPM1307-A | 0.25 | 64 | 2 | 0.5 | 8 | 8 | 4 | 0.5 | 64 |
| SPM1601 | 0.25 | 64 | 2 | 2 | 4 | 8 | 16 | 0.5 | 32 |
| SPM1604 | ≦0.06 | ≦0.06 | ≦0.06 | ≦0.06 | 4 | 1 | 4 | ≦0.06 | 16 |
| SPM1605 | 0.5 | 32 | 2 | 1 | 4 | 4 | 4 | 0.25 | 16 |
| SPM1606 | 0.12 | 0.25 | ≦0.06 | ≦0.06 | 0.12 | 2 | 4 | 0.25 | 16 |
| SPM1608 | ≦0.06 | 32 | 2 | 2 | 8 | 8 | 8 | 0.5 | 128 |

Bifidobacterium pseudocatenulatum

| | | | | | | | | | |
|---|---|---|---|---|---|---|---|---|---|
| KCTC3223 | 0.5 | 128 | 4 | 2 | 16 | 8 | 8 | 2 | >128 |
| SPM1204 | 0.5 | 128 | 2 | 2 | 2 | 8 | >128 | 2 | 128 |
| SPM1309 | 0.25 | 32 | 2 | 0.25 | 4 | 2 | 8 | 0.5 | >128 |

Bifidobacterium longum

| | | | | | | | | | |
|---|---|---|---|---|---|---|---|---|---|
| KCTC3128 | 0.5 | 128 | 4 | 2 | 16 | 8 | 4 | 1 | >128 |
| SPM1205 | ≦0.06 | 8 | 2 | 0.5 | 2 | 2 | 4 | 0.12 | 4 |
| SPM1206 | 0.12 | 8 | 2 | 1 | 4 | 1 | 8 | 0.25 | 32 |
| SPM1207 | 0.25 | 16 | 2 | 1 | 4 | 8 | >128 | 2 | 16 |

TIG: tigecyclin,
STR: streptomycin,
VAN: vancomycin,
TEC: teicoplanin,
DAP: daptomycin,
LNZ: linezolid,
CLI: clindamycin,
QDA: quinupristin/dalfopristin,
TOB: tobramycin

### Test Example 4

### Cellular Toxicity Assay of Lactobacillus

In order to check the cellular toxicity of a cellular extract of the Bifidobacterium spp. SPM separated in Example 1 or culture supernatant thereof, MTT assay was performed.

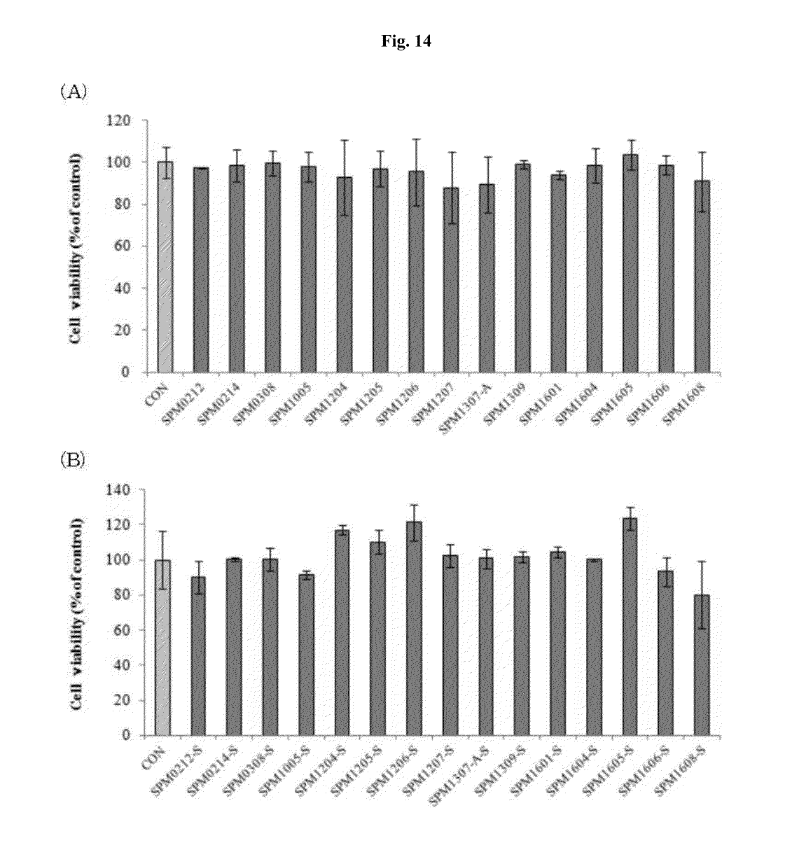

Extracts of lactobacillus of 1×10⁸ colony forming unit (CFU) and the culture supernatants thereof of various concentration were treated to the HepG 2 and HepG 2.2.15 cells cultured to the concentration of 1×10⁴ cells/well using a 96-well plate, and cultured for 24 hours. MTT reagent (5 mg/ml) 10 μl was added to each well, and reacted at an incubator of 37° C. and 5% CO₂ for 4 hours. After the reaction was completed, DMSO 100 μl was added, and the absorbance was measured at 570 nm using an ELISA reader. The results were listed in FIG. 14.

As shown in FIG. 14, both of the cellular extracts of the 15 kinds of lactobacillus strains separated and identified (FIG. 14 A) and the culture supernatants thereof (FIG. 14 B) did not significantly affect to the viabilities of the HepG2 and HepG2.2.15 cells.

### Test Example 5

### Separation and Analysis of Anti-Virus Active Material

In order to find out whether the antivirus active material of the bifidobacterium adolescentis (Bifidobacterium adolescentis) SPM0212 cell extract is a proteinaceous material or not, the extract of the cells of 1×10⁸ CFU was heated at 50° C. for 10 min, and the HBsAg ELISA was performed as described in Test Example 3. The absorbance was measured at 595 nm using Bio-Rad Protein assay (based on the method of Bradford) to find out the protein content in the cellular extract.

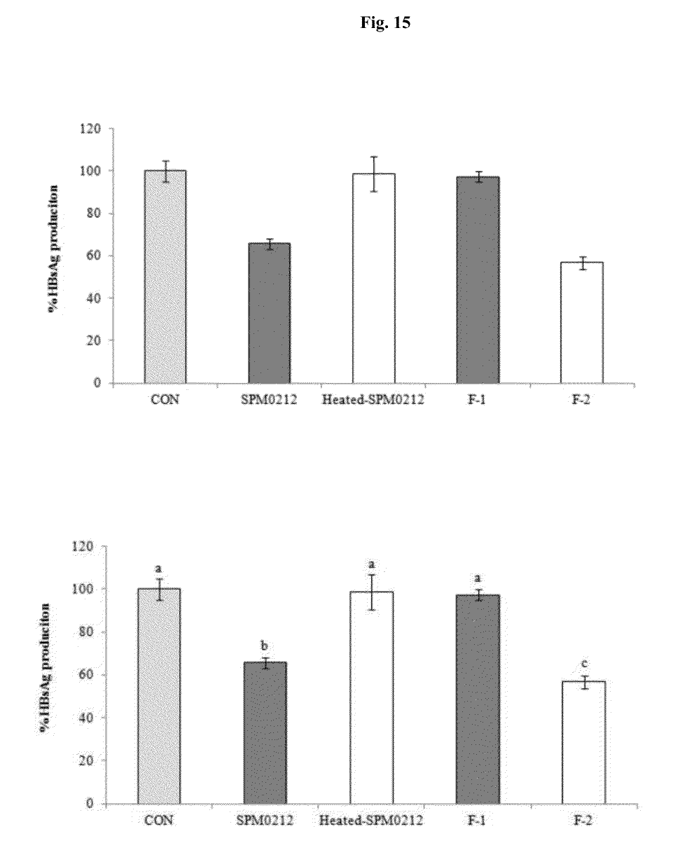

Further, in order to estimate molecular weight of the antivirus active material, the material was fractioned on the basis of 30 kDa, and HBsAg ELISA was performed using Amicon Ultra-0.5 Centrifugal Filter Devices (30,000 Nominal Molecular Weight Limit, NMWL) (Millipore, USA) as described in Test Example 3. The results were listed in FIG. 15.

The bifidobacterium adolescentis SPM0212 (1×10⁸ CFU) cell extract contained protein of 330.7 μg/mL, and as shown in FIG. 15, the heat-treated bifidobacterium adolescentis SPM0212 cell extract did not show the inhibitory effect on the HBsAg production. Further, this antiviral activity was only observed in the fraction of 30 kDa or less, and therefore, the antivirus active material of the bifidobacterium adolescentis SPM0212 cell extract was estimated as a proteinaceous material of molecular weight of 30 kDa or less.

While the invention has been described with respect to the above specific embodiments, it should be recognized that various modifications and changes may be made and also fall within the scope of the invention as defined by the claims that follow.

What is claimed is:

1. A method for preventing and treating hepatitis B comprising: administrating a pharmaceutical composition comprising the bifidobacterium adolescentis SPM0212 (Accession No.: KCTC 18120P) or culture thereof to a subject in need thereof.

2. The method of claim 1, further comprising, administrating lamivudine with the pharmaceutical composition comprising the bifidobacterium adolescentis SPM0212 (Accession No.: KCTC 18120P) or culture thereof to a subject in need thereof.

3. The method of claim 1, wherein administration of the pharmaceutical composition comprising the bifidobacterium adolescentis SPM0212 (Accession No.: KCTC 18120P) or culture inhibits production of antigen s of hepatitis B virus, and the expression of DNA, RNA and HBsAg of the hepatitis B virus in the subject in need thereof.
